(19) United States
(12) Patent Application Publication
(10) Pub. No.: US 2026/0295262 A1
(43) Pub. Date: Oct. 1, 2026

(54) TREATING CHRONIC COUGH USING SPINAL CORD STIMULATION

(71) Applicant: Spiro Medical, Inc., Irvine, CA (US)

(72) Inventor: Stephen T. PYLES, Ocala, FL (US)

(73) Assignee: Spiro Medical, Inc., Irvine, CA (US)

(21) Appl. No.: 19/656,856

(22) Filed: Apr. 23, 2026

Related U.S. Application Data
(63) Continuation of application No. 19/578,560, filed on Mar. 25, 2026.
(60) Provisional application No. 63/778,170, filed on Mar. 26, 2025.

(51) Int. Cl. A61N 1/36; A61N 1/05

(52) U.S. Cl. CPC A61N 1/3611; A61N 1/0551; A61N 1/36062; A61N 1/36157; A61N 1/36171; A61N 1/36175

(57) ABSTRACT

A device and method for treating chronic cough are disclosed. The device generates electrical pulses directed to a region of the spinal cord column. The electrical pulses provide a neuromodulation to mitigate chronic cough. The method includes acquiring a pulse generator, and programming the pulse generator to generate the electrical pulses directed to the region of the spinal cord to provide the neuromodulation to mitigate the chronic cough.

### CROSS REFERENCE TO RELATED APPLICATIONS

This application is a continuation application of U.S. application Ser. No. 19/578,560, filed Mar. 25, 2026, which claims priority to and the benefit of U.S. Provisional Patent Application No. 63/778,170 filed on Mar. 26, 2025. The foregoing applications are incorporated by reference herein in its entirety.

### BACKGROUND

Chronic cough is defined in adults as a cough that lasts for more than eight weeks. When intractable to treatment, it is defined as refractory chronic cough (RCC). Unexplained chronic cough (UCC) may be diagnosed when all other etiologies have been excluded. Chronic cough, and in particular UCC and RCC, result in a serious, often unrecognized, disease burden.

Patients with chronic cough report many physical and psychological effects, which impair their quality of life. Chronic cough also has a significant economic burden for patients and healthcare systems. Further, UCC and RCC diagnosis and treatment are often delayed for many years because a stepwise empirical therapeutic regimen is often recommended.

Evidence supporting most currently recommended treatments is limited. Many treatments do not address the underlying pathology, are used off-label, have limited efficacy, and produce significant side-effects, such that there are no approved treatments for chronic cough, UCC, and RCC. Therefore, there is a significant unmet need for a safe and effective therapy for chronic cough, UCC, and RCC that targets the underlying disease mechanisms, and/or that overrides the hyper-responsive cough reflex.

### SUMMARY

In general terms, the present disclosure relates to treating chronic cough by stimulating neural tissue proximate to and/or inside of a targeted region of the spinal column. In one possible configuration, a device is programmed to generate electrical pulses to provide a neuromodulation signal to mitigate chronic coughing. Various aspects are described in this disclosure, which include, but are not limited to, the following aspects.

One aspect relates to a device for treating chronic cough, the device comprising: a processing circuitry having non-transitory computer readable storage media storing instructions which, when executed by the processing circuitry, cause the processing circuitry to: generate electrical pulses directed to a region of the spinal cord column, the electrical pulses providing a neuromodulation to mitigate chronic cough.

Another aspect relates to a method of treating chronic cough, the method comprising: acquiring a pulse generator configured for neuromodulation; and programming the pulse generator to generate electrical pulses directed to a region of the spinal cord, the electrical pulses providing the neuromodulation to mitigate chronic cough.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system that can be used to mitigate chronic cough.

FIG. 2 illustrates a spine anatomy in which the system of FIG. 1 is implanted.

FIG. 3 illustrates examples of additional types of stimulators and leads that can be used to mitigate chronic cough.

FIG. 4 schematically illustrates an example of the system of FIG. 1 having a distal end of a lead implanted in proximity to neural tissue in a targeted region of the epidural space, and a proximal end of the lead plugged into a pulse generator.

FIG. 5 schematically illustrates another example of a stimulator of FIG. 3 having a distal end of a lead implanted in proximity to neural tissue in the targeted region of the epidural space.

FIG. 6 schematically illustrates another example of a stimulator of FIG. 3 having a distal end of a lead implanted in proximity to neural tissue in the targeted region of the epidural space.

FIG. 7 schematically illustrates an example of a method of treating chronic cough using the system of FIG. 1 or the stimulators of FIG. 3.

FIG. 8 schematically illustrates an example of a processing circuitry that can be included in the pulse generator of the system of FIG. 1, the stimulators of FIG. 3, and external controllers in FIGS. 4-6.

### DETAILED DESCRIPTION

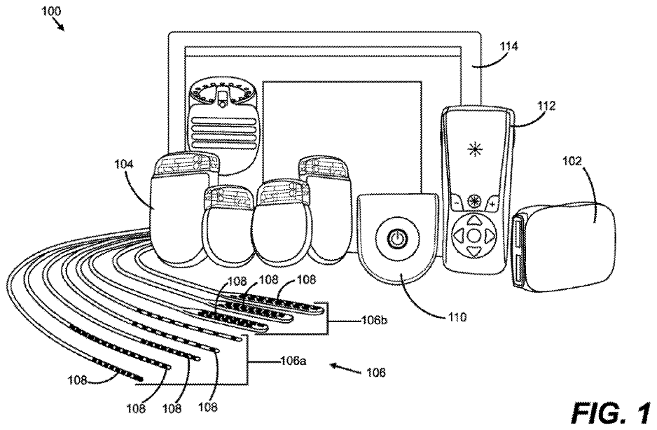

FIG. 1 illustrates an example of a system 100 that is designed to be implanted into a patient to deliver neuromodulation to tissue surrounding the spinal cord to mitigate chronic pain. As will be described, the system 100 can be programmed to additionally mitigate chronic cough. As used herein, the term chronic cough includes refractory chronic cough (RCC), unexplained chronic cough (UCC), asthma or allergy related cough, idiopathic cough, and any other type of chronic coughing.

Following a trial stimulation with an external trial stimulator 102, a pulse generator 104 is permanently implanted in the upper buttock or lower flank of a patient, and leads 106 are implanted within a targeted region of an epidural space of the spinal column. In this example, the leads 106 are designed to be physically connected to the pulse generator 104 to receive programmed electrical signals that are generated by the pulse generator 104. The leads 106 release the electrical pulses in the targeted region of the epidural space to provide neuromodulation of neural tissues to mitigate or eliminate chronic cough.

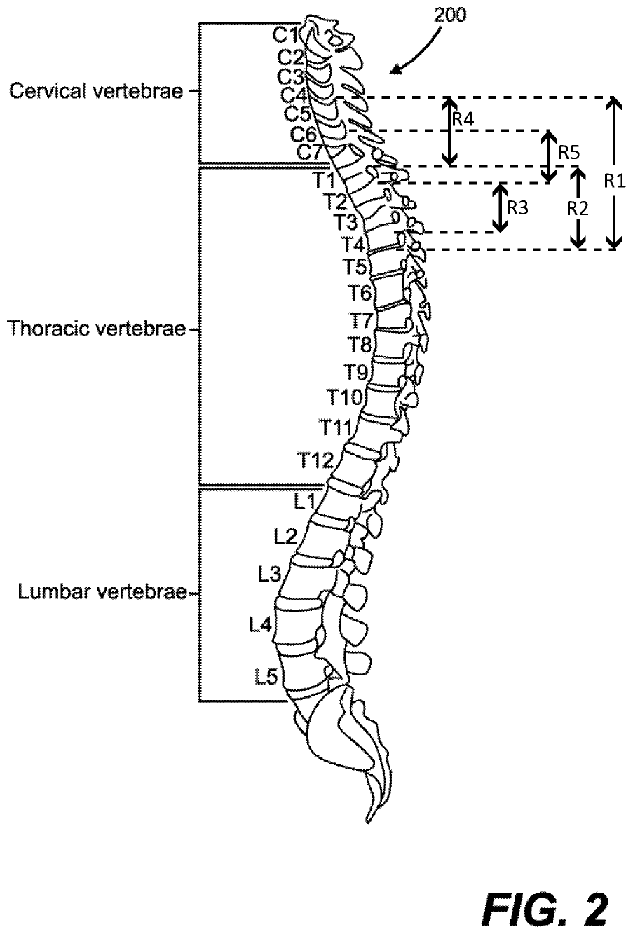

FIG. 2 illustrates a spine anatomy 200 in which the system 100 is implanted. Targeted regions of the epidural space for implanting the leads 106 are shown. A first targeted region R1 of the epidural space for implanting the leads 106 of the system 100 is between the C4 and T4 vertebrae. A second targeted region R2 of the epidural space for implanting the leads 106 is between the T1 and T4 vertebrae. A third targeted region R3 of the epidural space for implanting the leads 106 is between the T2 and T3 vertebrae. A fourth targeted region R4 of the epidural space for implanting the leads 106 is between the C4 and C7 vertebrae. A fifth targeted region R5 of the epidural space for implanting the leads 106 is between the C6 and T1 vertebrae.

In some examples, at least one lead 106 of the system 100 is implanted in the epidural space in the third targeted region R3 (i.e., between T2 and T4), and at least one lead 106 is implanted in the epidural space in the fourth targeted region R4 (i.e., between C4 and C7). In some examples, two of the leads 106 are implanted in the epidural space in the third targeted region R3 (i.e., between T2 and T4 vertebrae), and/or two of the leads 106 are implanted in the epidural space in the fourth targeted region R4 (i.e., between C4 and C7 vertebrae).

The targeted regions R1-R5 of the epidural space allow the neuromodulation provided by the system 100 to stimulate a dorsal spinal cord region. The neuromodulation targets parasympathetic and sympathetic ganglia in the targeted regions. The neuromodulation provided by the system 100 when programmed in accordance with the neuromodulation parameters described herein can mitigate or eliminate chronic cough without the need for drug intervention.

Referring back to FIG. 1, the system 100 can use percutaneous leads 106a that can be temporarily or permanently implanted inside the targeted regions R1-R5 of the epidural space. Alternatively, the system 100 can use permanent leads such as paddle leads 106b that are permanently implanted inside the targeted regions R1-R5 of the epidural space.

The percutaneous leads 106a and the paddle leads 106b each include electrical contacts 108 that can be cylindrical in the case of the percutaneous leads 106a, or that can be planar in the case of the paddle leads 106b. The electrical contacts 108 release the electrical pulses inside the targeted regions R1-R5 of the epidural space to provide neuromodulation that mitigates or even eliminates chronic cough without the need for drug intervention.

As an example, the system 100 can utilize the percutaneous leads 106a that have a quantity of 16 of the electrical contacts 108 at the distal ends thereof. The leads 106a, 106b plug into the pulse generator 104 at the proximal ends of the leads. For example, a torque wrench can be used to tighten set screws that lock the leads 106a, 106b into the pulse generator 104. Also, the system 100 can include lead splitters to connect multiple leads to the pulse generator 104.

The system 100 also includes multiple non-implantable components to aid with device setup prior to implantation, as well as communication between components following implantation. For example, the external trial stimulator 102 is used prior to permanent implantation of the pulse generator 104 to gauge the patient's response to spinal cord stimulation. The external trial stimulator 102 is intended to provide trial stimulation having identical stimulation capabilities as the pulse generator 104, to the implanted leads such that the neuromodulation parameters may be fine-tuned to fit the patient's specific needs.

To facilitate the tuning and adjustment of stimulation parameters, the system 100 includes a clinician programmer 114. In the example shown in FIG. 1, the clinician programmer 114 is a portable tablet computer, or similar type of computing device. The clinician programmer 114 is installed with programming software, and an optional programming wand may be used to allow the clinician programmer 114 to communicate wirelessly with the external trial stimulator 102 or the pulse generator 104. After implantation of the leads 106, providers will maintain control of a remote control 112. As shown in FIG. 1, the remote control 112 is a handheld, battery-operated unit that uses telemetry to communicate with the stimulators, allowing the provider to adjust and control the stimulation therapy (e.g., turn the SCS system on and off). The system 100 can include a charger 110 for transcutaneous charging of an internal power source (e.g., battery) of the pulse generator 104 once the pulse generator 104 is implanted inside the patient's body. The pulse generator may be programmed to cycle on and off. For example, the pulse generator may provide neuromodulation on the following cycles: 10 minutes on and 30 minutes off; 5 minutes on and 35 minutes off; 15 minutes on and 25 minutes off; or 20 minutes on and 20 minutes off.

The system 100 can include additional accessories such as stylets that can be used to maneuver the leads 106 through the epidural space to the desired implant location; a template that can be optionally used to guide the physician to create the correct sizing of a subcutaneous pocket for implanting the pulse generator 104; an insertion needle that can be used during the implantation procedure to introduce the percutaneous leads into the epidural space; a lead blank that can be optionally used during the implantation procedure to clear a path for the introduction of the leads 106 into the epidural space; a tunneling tool that can be used to create a subcutaneous tunnel from the implantation site of the pulse generator 104 to the implant location of the lead 106; connector plug/port plugs that can be used to seal the port(s) of the pulse generator 104 that are not in use; a cable extension that can be used to connect the leads 106 to the external trial stimulator 102 during intraoperative testing and trial phase; and external adaptors that can be used for connecting other types of leads to external stimulators during in-office evaluation.

The release of the electrical pulses by the electrical contacts 108 of the leads 106 when implanted inside the epidural space causes neuromodulation of the dorsal column, which is responsible for the regulation of pain. The neuromodulation overrides the dysregulation of the spinothalamic tract by suppressing neurons within the dorsal horn of the spinal cord using the electrical pulses that can be distributed in a variety of patterns. The neuromodulation is able to mitigate chronic intractable pain of the trunk and/or limbs. Depending on where the electrical contacts 108 of the leads 106 are positioned in the epidural space, the neuromodulation can mitigate unilateral or bilateral pain associated with failed back surgery syndrome; complex regional pain syndrome (CRPS) Types I and II; intractable lower back pain and leg pain with or without prior back surgery; diabetic peripheral neuropathy of the lower extremities; radicular pain syndrome; radiculopathies resulting in pain secondary to failed back syndrome or herniated disc; epidural fibrosis; degenerative disc disease (herniated disc pain refractory to conservative and surgical interventions); arachnoiditis; multiple back surgeries.

Vagal parasympathetic innervation of airways plays a pivotal role in the onset of symptoms associated with inflammatory airway diseases like asthma and COPD. Different mediators released during inflammation, such as interleukin (IL)-4, interleukin (IL)-5, and interleukin (IL)-13, can activate and sensitize both peripheral and central parasympathetic neurons resulting in bronchoconstriction and increased mucus production. Suppression of inflammation using steroids or antibodies targeting cytokines, IgE, and specific cells, is typically not sufficient as neuronal sensitization may be long-lasting and becomes independent leading to limited efficacy. Clinical data suggests that neuromodulation of specific sympathetic neurons by the system 100 can more effectively treat asthma and asthma-related chronic cough by causing both bronchodilation and blocking parasympathetic activation and sensitization by immune mediators.

For example, the neuromodulation provided by the system 100 can also effectively activate the sympathetic efferent nerves to cause bronchodilation through beta-adrenergic receptors which are abundant on airway smooth muscles. The neuromodulation also inhibits the parasympathetic nerve fibers to prevent bronchoconstriction through both afferent signaling and blocking the efferent fibers. Further, it is contemplated that the neuromodulation targets the visceral afferent nerves in the intermediate zone of the spinal cord which is the region of gray matter that lies between the posterior (or dorsal) and anterior (or ventral) horns of the spinal cord, and includes the rexed lamina X. When the electrical contacts 108 are implanted inside the targeted regions of the epidural space, the neuromodulation provided by the system 100 activates the sympathetic pulmonary axis which inhibits the parasympathetic innervation of the bronchial smooth muscles, preventing constriction of the bronchial smooth muscles. Also, the neuromodulation directly activates the sympathetic nerve fibers to activate beta-adrenergic receptors further supporting relaxation of bronchial smooth muscles. Thus, through stimulation of the dorsal spinal cord region between C4 and T4 vertebrae (or in some instances between the T1 and T4 vertebrae, or in some further instances between the T2 and T3 vertebrae, or between the C4 and C7 vertebrae, or in some instances between the C6 and T1 vertebrae), the system 100 can both maintain bronchodilation and block the upper airway hyper-responsiveness to various triggers thereby mitigating or even eliminating chronic cough.

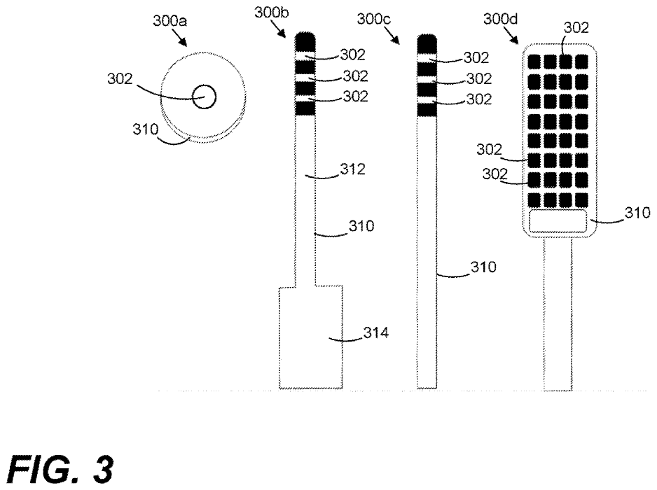

FIG. 3 illustrates examples of additional types of stimulators 300a-300d that can be used to mitigate or even eliminate chronic cough. The stimulators 300a-300d can be placed percutaneously, minimally invasively, or via open surgery. The stimulators 300a-300d release electrical pulses to stimulate neural tissue including sympathetic ganglia proximate the targeted regions R1-R4 of the epidural space of the spinal column (see FIG. 2).

The stimulators 300a-300d do not physically connect or disconnect to a separate pulse generator such as the pulse generator 104 shown in FIG. 1. Instead, the stimulators 300a-300d can each include an internal power source that generates the electrical pulses for release in the targeted regions R1-R4 of the epidural space. The internal power source housed on the stimulators 300a-300d can include one or more rechargeable batteries that can be recharged through wireless energy transfer from an external power source like the charger 110 (see FIG. 1).

Alternatively, the stimulators 300a-300d can wirelessly receive the electrical pulses from the external power source for releasing the electrical pulses in the targeted regions R1-R4 of the epidural space. Examples of wireless energy transfer for recharging the internal power source of the stimulators 300a-300d and/or for transferring the electrical pulses generated by the external power source to the stimulators 300a-300d can include inductance, midfield radiofrequency (RF), far-field RF, high frequency electromagnetic coupling, ultrasound energy transfer (UET), hypersound, and other wireless power communication techniques.

The stimulators 300a-300d each include one or more electrical contacts 302 mounted on a body 310. The elongated body may take the form of a lead. The electrical contacts 302 generate an electric field to stimulate the dorsal spinal cord region. The electrical contacts 302 may be cylindrical, semi-cylindrical, spherical, semi-spherical, or planar. In some examples, the electrical contacts 302 can be positioned along a length between a distal end and a proximal end of the body 310 that correlates with a length of a targeted region of the epidural space such as between C4 and T4 vertebrae, or between the T1 and T4 vertebrae, or between the T2 and T3 vertebrae, or between the C4 and C7 vertebrae, or in some further instances between the C6 and T1 vertebrae.

The stimulator 300a includes a body 310 that is disc shaped. In this example, the stimulator 300a is implantable via open surgery outside of the epidural space, but proximate to a targeted region of the epidural space. In this manner, the electrical pulses released by the stimulator 300a can stimulate sympathetic ganglia including spinal nerve roots and/or dorsal root ganglion (DRG) next to the targeted region of the epidural space, but not from inside the epidural space. The stimulator 300a can house an internal power source for generating the electrical pulses. The internal power source can include a rechargeable battery.

The stimulator 300b includes a body 310 that has tubular portion 312 and a housing portion 314 for housing components such as an internal power source. The tubular portion 312 is implantable inside the epidural space, while the housing portion 314 can remain outside of the epidural space. The body 310 of the stimulator 300b including the housing portion 314 can fold for deployment through a catheter such as an introducer sheath or epidural needle, and can then unfold once the tubular portion 312 of the body 310 is implanted inside the epidural space.

The stimulator 300c includes a body 310 that is tubular along its entire length. The stimulator 300c is implantable percutaneously through an introducer sheath or epidural needle. In some examples, the stimulator 300c is a temporary percutaneous implant, such as for conducting a spinal cord stimulation trial. Alternatively, the stimulator 300c can be a permanent percutaneous implant, such as after completion of a successful spinal cord stimulation trial. The stimulator 300c can house an internal power source for generating the electrical pulses, or can receive the electrical pulses from an external power source.

The stimulator 300d is an example of a device that includes paddle shaped lead. The stimulator 300d can be permanently implanted either via a minimally invasive procedure or via open surgery. Like in the examples described above, the stimulator 300c can house an internal power source for generating the electrical pulses. Alternatively, the stimulator 300c can receive the electrical pulses from an external power source to provide the neuromodulation in a targeted region of the epidural space.

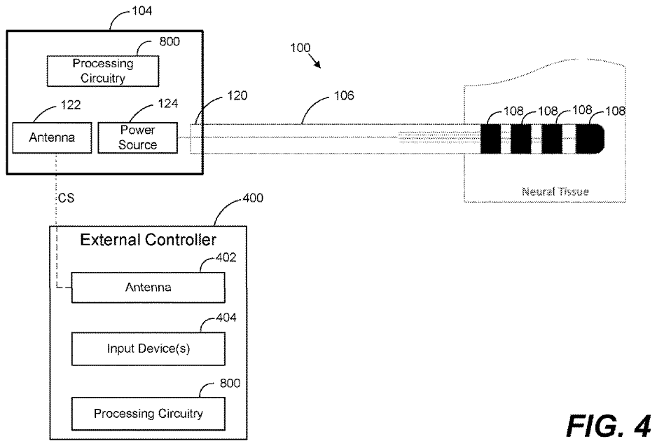

FIG. 4 schematically illustrates an example of the system 100 having a distal end of the lead 106 implanted in proximity to neural tissue in a targeted region R of the epidural space, and a proximal end of the lead 106 plugged into the pulse generator 104. An external controller 400 wirelessly communicates with the pulse generator 104 to program the pulse generator 104 to generate the electrical pulses to have optimal parameters to mitigate or eliminate chronic cough. The electrical pulse generated by the pulse generator 104 are programmed such that they are sub-perception such that there is no sensible artifact or side-effect felt by the patient.

As shown in FIG. 4, the system 100 includes the pulse generator 104, the lead 106 having electrical contacts 108 implanted in the epidural space, and the external controller 400. In this example, the lead 106 is plugged into a receptacle 120 of the pulse generator 104 such that the lead 106 is mechanically connected to the pulse generator 104. As described above, the pulse generator 104 generates the electrical pulse for transmission through the lead 106. The electrical contacts 108 release the electrical pulses to neuromodulate the neural tissue in a targeted region of the epidural space such as between the C4 and T4 vertebrae, or between the T1 and T4 vertebrae, or between the T2 and T3 vertebrae, or between the C4 and C7 vertebrae, or in some further instances between the C6 and T1 vertebrae.

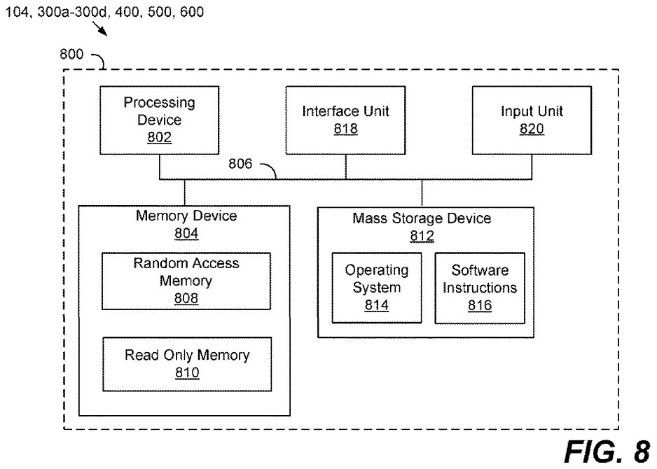

The pulse generator 104 includes an antenna 122, a power source 124, and a processing circuitry 800 that can include non-transitory computer readable storage media for storing parameters such as an amplitude, a frequency, and a pulse width that are optimal for mitigating or eliminating chronic cough. An example of the processing circuitry 800 is shown in FIG. 8, which is described in more detail further below.

In some examples, the antenna 122 is a passive antenna such that it only receives transmissions from the external controller 400. In alternative examples, the antenna 122 is an active antenna allowing two-way communications between the pulse generator 104 and the external controller 400 such as to share data, control signals, and other transmissions.

The external controller 400 includes a processing circuitry 800 operatively connected to an antenna 402 and one or more input devices 404. The antenna 402 wirelessly communicates with the antenna 122 of the pulse generator 104 to program the pulse generator 104 to generate the electrical pulses to have the predetermined combination of the amplitude, the frequency, and the pulse width to mitigate or eliminate chronic cough.

The antenna 402 can transmit control signals CS for programming the pulse generator 104 based on the inputs received by the one or more input devices 404. The one or more input devices 404 can include push buttons and/or a touchscreen display such as when the external controller 400 is a tablet computer like the one shown in FIG. 1. The antenna 402 can wirelessly communicate with the antenna 122 of the pulse generator 104 via Bluetooth, Wi-Fi, Zigbee, near-field communication (NFC), or other wireless communications protocols.

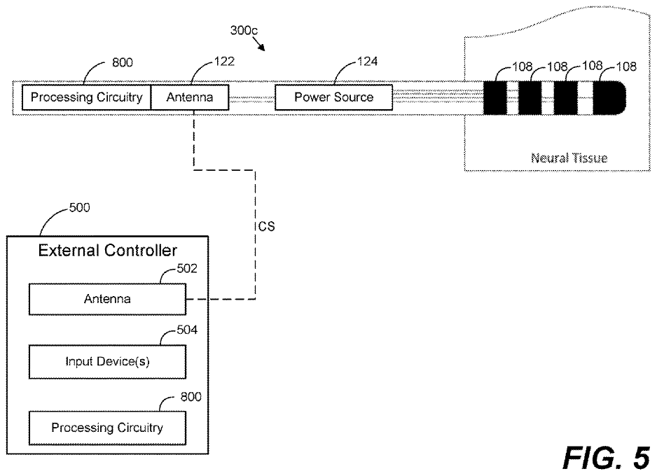

FIG. 5 schematically illustrates another example of the stimulator 300c having a distal end implanted in proximity to neural tissue in a targeted region of the epidural space. In this example, the stimulator 300c is a leadless stimulator because the stimulator 300c does not include leads attached to a pulse generator. An external controller 500 wirelessly communicates with the stimulator 300c to program the stimulator 300c to generate the electrical pulses to have optimal parameters to mitigate or eliminate chronic cough.

In FIG. 5, the stimulator 300c includes electrical contacts 108 implanted in the neural tissue. The stimulator 300c for includes a power source 124 that generates electrical pulses. The electrical contacts 108 release the electrical pulses to neuromodulate the neural tissue in the targeted region of the epidural space such as between the C4 and T4 vertebrae, or between the T1 and T4 vertebrae, or between the T2 and T3 vertebrae, or between the C4 and C7 vertebrae, or in some further instances between the C6 and T1 vertebrae.

The stimulator 300c further includes an antenna 122 and a processing circuitry 800 that can include non-transitory computer readable storage media that stores neuromodulation parameters such as the predetermined combination of the amplitude, the frequency, and the pulse width that are optimal for mitigating or eliminating chronic cough.

In some examples, the antenna 122 is a passive antenna such that it only receives transmissions from the external controller 500. In alternative examples, the antenna 122 is an active antenna allowing two-way communications between the stimulator 300c and the external controller 500 such as to share data, control signals, and other transmissions.

The external controller 500 includes a processing circuitry 800 operatively connected to an antenna 502 and one or more input devices 504. The antenna 502 wirelessly communicates with the antenna 122 of the stimulator 300c to program the stimulator 300c to generate the electrical pulses to have the predetermined combination of the amplitude, the frequency, and the pulse width to mitigate or eliminate chronic cough.

The antenna 502 can transmit control signals CS for programming the stimulator 300c based on the inputs received by the one or more input devices 504. The one or more input devices 504 can include push buttons and/or a touchscreen display such as when the external controller 500 is a tablet computer like the one shown in FIG. 1. The antenna 502 can wirelessly communicate with the antenna 122 of the stimulator 300c via Bluetooth, Wi-Fi, Zigbee, near-field communication (NFC), or other wireless communications protocols.

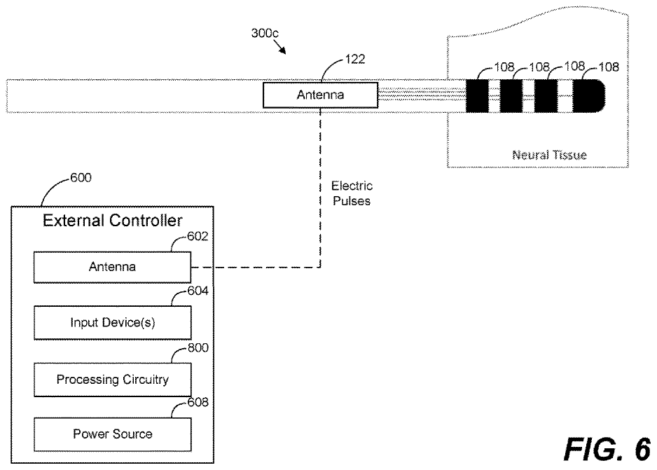

FIG. 6 schematically illustrates another example of the stimulator 300c having a distal end implanted in proximity to neural tissue in a targeted region of the epidural space. In this example, the stimulator 300c is another example of a leadless stimulator because the stimulator 300c does not include leads attached to a pulse generator. An external controller 600 wirelessly communicates with the stimulator 300c to control the stimulator 300c to release the electrical pulses having the neuromodulation parameters to mitigate or eliminate chronic cough.

In FIG. 6, the stimulator 300c includes electrical contacts 108 implanted in proximity to the epidural neural tissue. The stimulator 300c does not include an internal power source. Instead, an external power source 608 is included on the external controller 600. The external power source 608 can include one or more batteries such as rechargeable or disposable batteries. The external power source 608 generates the electrical pulses for transfer to the stimulator 300c for release by the electrical contacts 108 to neuromodulate the neural tissue in the targeted region of the epidural space such as between the C4 and T4 vertebrae, or between the T1 and T4 vertebrae, or between the T2 and T3 vertebrae, or between the C4 and C7 vertebrae, or in some further instances between the C6 and T1 vertebrae.

In some examples, the stimulator 300c receives subcutaneous energy from an external device such as a transcutaneous electrical nerve stimulation (TENS) unit or similar type of device. The energy is transmitted through the skin toward a proximal end of the stimulator 300c, and then the conducted energy is transferred to the electrical contacts 108 at a distal end or tip of the stimulator 300c for stimulating a neural target of the spinal nerves and/or DRG.

The external controller 600 includes an antenna 602 that can transfer the electrical pulses generated by the external power source 608 to the antenna 122 on the stimulator 300c for release by the electrical contacts implanted in proximity to the targeted region of the epidural space. The electrical pulses can be transferred from the antenna 602 to the antenna 122 using the wireless energy transfer techniques described above such as inductance, midfield radiofrequency (RF), far-field RF, high frequency electromagnetic coupling, ultrasound energy transfer (UET), hypersound, and other wireless energy transfer techniques. In this example, the antenna 122 is a passive antenna such that it only receives transmissions from the external controller 600.

The external controller 600 includes a processing circuitry 800 operatively connected to an antenna 602, one or more input devices 604, and the external power source 608. The processing circuitry 800 controls the external power source 608 to generate the electrical pulses to have the predetermined combination of the amplitude, the frequency, and the pulse width to mitigate or eliminate chronic cough based on the neuromodulation parameters selected or entered by a user of the external controller 600.

Table 1 provides value ranges for the neuromodulation parameters that are used to mitigate or eliminate chronic cough by the electrical pulses released by the electrical contacts 108 positioned proximate to and/or inside of the targeted region of the epidural space between the C4 and T4 vertebrae, or between the T1 and T4 vertebrae, or between the T2 and T3 vertebrae, or between the C4 and C7 vertebrae. The neuromodulation parameters include an amplitude that defines a strength of the electrical pulses delivered to the neural tissue in the targeted region, a pulse width that defines a duration of the electrical pulses, and a frequency that defines how often the electrical pulses are delivered to the neural tissue in the targeted region.

TABLE 1

| Parameter | Value Range |
|---|---|
| Amplitude (mAmp) | 0.5-5.0 |
| Frequency (Hz) | 250-500 |
| Pulse Width (μs) | 200-500 |

The external controllers 400-600 can be used to program the pulse generator 104 of the system 100 and the stimulators 300a-300d to generate the electrical pulses having the amplitude, frequency, and pulse width values defined in Table 1. The amplitude, frequency, and pulse width values defined in Table 1 can be stored on non-transitory computer readable storage media housed on the pulse generator 104 of the system 100. Additionally, the amplitude, frequency, and the pulse width values defined in Table 1 can be stored on non-transitory computer readable storage media housed on the stimulators 300a-300d. The pulse generator 104 and the power sources 124 can then generate the electrical pulses having the amplitude, frequency, and pulse width values defined in Table 1 for delivery to the neural tissue including the sympathetic ganglia in the targeted region of the epidural space.

In some examples, prior to programming the pulse generator 104 of the system 100 and the stimulators 300a-300d to generate the electrical pulses having the amplitude, frequency, and pulse width values defined in Table 1, a test procedure can be performed to confirm stimulation of the chest area. The test procedure can be performed when implanting the external trial stimulator 102 such as for a trial stimulation. The test procedure can include controlling the external trial stimulator 102 to emit electrical pulses having a low frequency of about 30 Hz to about 50 Hz, a pulse width of about 150 μs to about 300 μs, and then gradually increasing the amplitude starting from 0.5 mAmp until a perception threshold is reached such that the patient feels a tingling or vibration sensation (similar to that of paresthesia) in the chest area. Once the patient confirms the tingling or vibration sensation in the chest area, the external trial stimulator 102 can be programmed to have the parameters defined in Table 1 such that the spinal cord stimulation for mitigating or eliminating chronic cough is sub-perception.

In examples where the stimulators 300a-300d do not include an internal power source, the amplitude, frequency, and pulse width values defined in Table 1 can be stored on non-transitory computer readable storage media housed on the external controller 600. The processing circuitry 800 of the external controller 600 controls the external power source 608 to generate the electrical pulse having the amplitude, frequency, and pulse width values defined in Table 1. The electrical pulses are transferred from the external controller 600 to the stimulators 300a-300d for delivery by the electrical contacts 108 to the neural tissue including the sympathetic ganglia in the targeted region of the epidural space.

In some examples, the pulse generator 104 and the stimulators 300a-300d are programmed to cycle the neuromodulation on and off for predetermined periods of time. As an example, the pulse generator 104 and the stimulators 300a-300d are programmed to repeatedly cycle the neuromodulation for 10 minutes on and 20 minutes off. Cycling the neuromodulation on and off conserves energy, and thereby prolongs the life of the power sources 124, 608.

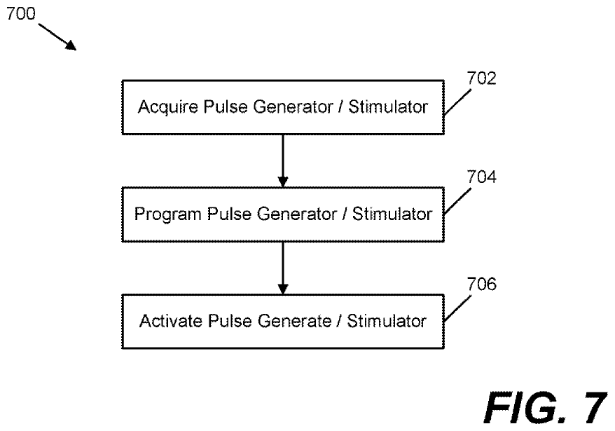

FIG. 7 schematically illustrates an example of a method 700 of treating chronic cough using spinal cord stimulation. The method 700 can include using the system 100 of FIG. 1 or the stimulators 300a-300d of FIG. 3. The method 700 improves efficacy of treating chronic cough over current treatments that are generally unsatisfactory. A further advantage of the method 700 includes treating chronic cough without drug intervention, and thereby avoiding the need to take medications such as neuromodulators which can be expensive and can have side effects.

The method 700 includes an operation 702 of acquiring the pulse generator 104 which, as described above, is configured for spinal cord stimulation. Alternatively, operation 702 can include acquiring one of the stimulators 300a-300d which are also configured for spinal cord stimulation. As discussed above, the pulse generator 104 and the stimulators 300a-300d are generally configured to provide spinal cord stimulation to mitigate chronic cough in patients.

The method 700 includes an operation 704 of programming the pulse generator 104 of the system 100 shown in FIG. 1 to generate electrical pulses to provide a neuromodulation that mitigates or eliminates chronic cough. Alternatively, operation 704 can include programming the stimulators 300a-300d shown in FIG. 3 to generate the electrical pulses to provide the neuromodulation that mitigates or eliminates chronic cough.

In operation 704, the pulse generator 104 and the stimulators 300a-300d are programmed to generate the electrical pulses for providing neuromodulation to a target region of the epidural space. The neuromodulation is programmed to stimulate a dorsal spinal cord region. The pulse generator 104 and the stimulators 300a-300d are programmed to generate the electrical pulses for targeting sympathetic ganglia between the C4 and T4 vertebrae, or between the T1 and T4 vertebrae, or between the T2 and T3 vertebrae, or between the C4 and C7 vertebrae.

In operation 704, the pulse generator 104 and the stimulators 300a-300d are programmed to provide the neuromodulation having parameters that include an amplitude that ranges from about 0.5 mAmp to about 5.0 mAmp, a predefined frequency that ranges from about 250 Hz to about 500 Hz, and a predefined pulse width that ranges from about 200 μs to about 500 μs. As discussed above, the values of the amplitude, the frequency, and the pulse width can mitigate or eliminate chronic cough without drug intervention.

In some examples, the method 700 can include an operation 706 of activating the pulse generator 104 after implantation inside a patient. Alternatively, operation 706 of activating one of the stimulators 300a-300d after implantation inside a patient. Activating the pulse generator 104 and the stimulators 300a-300d causes these devices to provide the neuromodulation in accordance with the programming done in operation 704.

Chronic cough, which is often characterized by many triggers that present themselves in everyday environments, significantly impacts patients' quality of life and limits their ability to engage in routine activities. The system 100 and stimulators 300a-300d when programmed for the treatment of chronic cough as disclosed herein can mitigate these challenges by providing patients with greater freedom and flexibility in managing their chronic cough. With these implantable devices, patients may experience reduction or elimination of all coughing and reduced susceptibility to environmental triggers, allowing them to pursue a drug-free and more fulfilling and unrestricted lifestyle. By alleviating the need for medications that typically do not work for a majority of patients, and constant interruption of normal life activities, along with the avoidance of chronic triggers, the system 100 and stimulators 300a-300d when programmed for the treatment of chronic cough as disclosed herein empowers patients to reclaim control over their health and enjoy a higher quality of life and get back to their daily lives.

The amplitude, the frequency, and the pulse width values for the neuromodulation to eliminate chronic cough are unexpected because spinal cord stimulation has been used to produce or restore coughing for spinal cord injury patients, which teaches away from using spinal cord stimulation to eliminate chronic cough. Using spinal cord stimulation to restore coughing is evidenced by U.S. Pat. No. 8,751,004 B2, entitled Bipolar Spinal Cord Stimulation to Activate the Expiratory Muscles to Restore Cough, granted on Jun. 10, 2014, and further by U.S. Pat. No. 5,999,855, entitled Method and Apparatus for Electrical Activation of the Expiratory Muscles to Restore Cough, granted on Dec. 7, 1999.

FIG. 8 schematically illustrates an example of the processing circuitry 800 that can be used to implement various aspects described herein including functions performed on the pulse generator 104, the stimulators 300a-300d, and the external controllers 400-600. The processing circuitry 800 includes a processing device 802 and a memory device 804. Examples of the processing device 802 include a processor, a processing unit, a central processing unit (CPU), a microprocessor, a digital signal processor, a field-programmable gate array, and the like.

The memory device 804 can include a random-access memory (RAM) 808 and a read-only memory (ROM) 810. Basic input and output logic having routines to transfer data between elements in the processing circuitry 800 can be stored in the ROM 810.

In some examples, the processing circuitry 800 can include a mass storage device 812. The mass storage device 812 is connected to the processing device 802 through the system bus 806. The memory device 804 and the mass storage device 812 are examples of computer-readable data storage media that provides non-volatile, non-transitory storage for the processing circuitry 800. The memory device 804 and the mass storage device 812 can store an operating system 814, software instructions 816, and/or data for execution by the processing device 802.

The computer-readable data storage media can be any available non-transitory, physical device or article of manufacture from which the processing circuitry 800 can read data and/or instructions. Computer-readable data storage media can include removable and non-removable media implemented in any method or technology for storage of information such as computer-readable software instructions, data structures, program modules or other data. Types of computer-readable data storage media include, but are not limited to, RAM, ROM, EPROM, EEPROM, flash memory or other solid-state memory technology, or any other medium which can be used to store information, and which can be accessed by the processing device 802.

The processing circuitry 800 can operate in a networked environment using logical connections to other devices. For example, the processing circuitry 800 can connect to another device through an interface unit 818. The interface unit 818 can connect to additional systems and devices through wired connections such as Ethernet, or through Bluetooth, Wi-Fi, Zigbee, and other wireless technologies. The processing circuitry 800 can also include an input unit 820 for receiving and processing inputs from input devices such as the external controller 400.

The mass storage device 812 and the RAM 808 can store software instructions and data. The software instructions can include an operating system 814 suitable for controlling the processing circuitry 800. The mass storage device 812 and/or the RAM 808 can also store the software instructions 816, which when executed by the processing device 802, cause the processing device 802 to provide the functionalities disclosed herein.

The various embodiments described above are provided by way of illustration only and should not be construed to be limiting in any way. Various modifications can be made to the embodiments described above without departing from the true spirit and scope of the disclosure.

What is claimed is:

1. A method of treating at least one of refractory chronic cough (RCC), unexplained chronic cough (UCC), and idiopathic cough, the method comprising:
acquiring a pulse generator configured for neuromodulation; and
programming the pulse generator to generate electrical pulses directed to a region of the spinal cord, the electrical pulses providing the neuromodulation to mitigate at least one of refractory chronic cough (RCC), unexplained chronic cough (UCC), and idiopathic cough.

2. The method of claim 1, wherein the electrical pulses are programmed to have an amplitude ranging from about 0.5 mAmp to about 5.0 mAmp, a frequency ranging from about 250 Hz to about 500 Hz, and a pulse width ranging from about 200 μs to about 500 μs.

3. The method of claim 1, further comprising:
activating the pulse generator after implantation inside a patient.

4. The method of any of claim 1, further comprising:
providing the neuromodulation to stimulate a dorsal spinal cord region to mitigate or eliminate the at least one of refractory chronic cough (RCC), unexplained chronic cough (UCC), and idiopathic cough without drug intervention.

5. The method of any of claim 1, further comprising:
providing the neuromodulation to target parasympathetic and sympathetic ganglia between the C4 and T4 vertebrae.

6. The method of any of claim 1, further comprising:
providing the neuromodulation to target parasympathetic and sympathetic ganglia between the T1 and T4 vertebrae or between the C4 and C7 vertebrae.

7. The method of any of claim 1, further comprising:
providing the neuromodulation to target parasympathetic and sympathetic ganglia between the T2 and T3 vertebrae.

8. The method of any of claim 1, further comprising:
programming the pulse generator to cycle the neuromodulation on and off.

9. A method of treating at least one of refractory chronic cough (RCC), unexplained chronic cough (UCC), and idiopathic cough, the method comprising:
delivering electrical pulses to a targeted region of an epidural space of the patient via one or more implanted electrical contacts, wherein the targeted region is located between the C4 and T4 vertebrae;
wherein the electrical pulses are defined by an amplitude ranging from 0.5 mAmp to 5.0 mAmp, a frequency ranging from 250 Hz to 500 Hz, and a pulse width ranging from 200 μs to 500 μs; and
wherein the electrical pulses provide neuromodulation that mitigates the at least one of refractory chronic cough (RCC), unexplained chronic cough (UCC), and idiopathic cough.

10. The method of claim 9, wherein delivering the electrical pulses comprises providing the neuromodulation at a sub-perception level such that the patient does not perceive a sensible artifact or side-effect.

11. The method of claim 10, further comprising, prior to delivering the sub-perception neuromodulation:
delivering test electrical pulses at a test frequency ranging from 30 Hz to 50 Hz and a test pulse width ranging from 150 μs to 300 μs; and
gradually increasing an amplitude of the test electrical pulses until the patient perceives a tingling or vibration sensation in a chest area to confirm stimulation targeting.

12. The method of claim 9, wherein providing the neuromodulation activates sympathetic efferent nerves to cause bronchodilation and inhibits parasympathetic nerve fibers to prevent bronchoconstriction.

13. The method of claim 9, wherein providing the neuromodulation targets nerves in an intermediate zone of the spinal cord that includes the rexed lamina X.

14. The method of claim 9, wherein the electrical contacts are of a leadless implant.

15. A method of treating at least one of refractory chronic cough (RCC), unexplained chronic cough (UCC), and idiopathic cough, the method comprising:
delivering electrical pulses to a targeted region of an epidural space of the patient via one or more implanted electrical contacts, wherein the targeted region is located between the C4 and T4 vertebrae;
wherein the electrical pulses are defined by a frequency ranging from 250 Hz to 500 Hz; and
wherein the electrical pulses provide neuromodulation that mitigates the at least one of refractory chronic cough (RCC), unexplained chronic cough (UCC), and idiopathic cough.

16. The method of claim 15, wherein the electrical pulses are further defined by an amplitude ranging from 0.5 mAmp to 5.0 mAmp and a pulse width ranging from 200 μs to 500 μs.

17. The method of claim 15, wherein delivering the electrical pulses comprises providing the neuromodulation at a sub-perception level such that the patient does not perceive a sensible artifact or side-effect.

18. The method of claim 15, wherein the targeted region of the epidural space is located between the T1 and T4 vertebrae or between the T2 and T3 vertebrae.

19. The method of claim 15, wherein providing the neuromodulation targets visceral afferent nerves in an intermediate zone of a spinal cord of the patient that includes a rexed lamina X.

20. The method of claim 15, further comprising, prior to delivering the electrical pulses to provide the neuromodulation:
delivering test electrical pulses at a test frequency ranging from 30 Hz to 50 Hz and a test pulse width ranging from 150 μs to 300 μs; and
gradually increasing an amplitude of the test electrical pulses until the patient perceives a tingling or vibration sensation in a chest area of the patient to confirm stimulation targeting.
